(19) United States
(12) Patent Application Publication
(10) Pub. No.: US 2019/0290887 A1
(43) Pub. Date: Sep. 26, 2019

(54) MEDICAL TUBE SECURING JIG

(71) Applicant: SUN MEDICAL TECHNOLOGY RESEARCH CORPORATION, Nagano (JP)

(72) Inventor: Kiyoto AYUZAWA, Nagano (JP)

(21) Appl. No.: 16/361,204

(22) Filed: Mar. 22, 2019

(30) Foreign Application Priority Data
Mar. 23, 2018 (JP) 2018-057223
Feb. 15, 2019 (JP) 2019-025939

(51) Int. Cl. A61M 25/02

(52) U.S. Cl. CPC A61M 25/02; A61M 2025/028; A61M 2025/0233

(57) ABSTRACT

A medical tube securing jig includes: a skin securing member having a flange portion and a protruding body; and a medical tube holding mechanism part to hold the medical tube. A recessed portion is formed on the protruding body and has female threads. The medical tube holding mechanism part includes: a sealing member elastically deformed by a pressing force applied to the sealing member when the sealing member engages with the recessed portion by fitting engagement thus being brought into close contact with a side wall surface of the recessed portion, a bottom surface of the recessed portion and the medical tube; and a sealing member pressing body having male threads and configured to apply the pressing force to the sealing member in fastening by making the male and female threads engage, and a slit portion is formed in the sealing member.

### TECHNICAL FIELD

The present invention relates to a medical tube securing jig for securing a medical tube at a transdermal part.

### BACKGROUND ART

With respect to a medical tube securing jig which is used for securing a medical tube at a transdermal part in inserting a medical tube into the inside of a body from the outside of the body, various types of medical tube securing jigs have been proposed. As such a medical tube securing jig, a transdermal catheter retaining set described in patent literature 1 can be exemplified, for example.

FIG. 16 is a cross-sectional view schematically showing a transdermal catheter retaining set 900 described in patent literature 1. As shown in FIG. 16, the transdermal catheter retaining set 900 described in patent literature 1 includes: a transdermal terminal 920 which has a through hole 921 for allowing a catheter 910 to pass therethrough and a recessed portion 922 having an opening of a diameter larger than a diameter of the through hole 921, and which has male threads 923 on an outer peripheral surface thereof; an annular rubber elastic body 940 which holds the catheter 910 by engaging with the recessed portion 922 of the transdermal terminal 920 by fitting engagement; a hard tube 950 which is annularly mounted on a portion of the catheter 910 held by the annular rubber elastic body 940; and a cap 960 which has female threads 961 threadedly engaged with the male threads 923 of the transdermal terminal 920 and has a through hole 963 for allowing the catheter 910 to pass therethrough at a center portion of an upper surface 962. The through hole 963 formed on the upper surface 962 of the cap 960 is formed of a tapered hole having a diameter increased toward the inside. Accordingly, the through hole 963 is expressed as “tapered hole 963”.

In assembling the transdermal catheter retaining set 900 having such a configuration, first, the cap 960 is a set in a free state (a state where the female threads 961 do not threadedly engage with the male threads 923 of the transdermal terminal 920), and the catheter 910 is made to pass through the center hole formed in the annular rubber elastic body 940 and the through hole 921 formed in the transdermal terminal 920. In such a configuration, the hard tube 950 annularly mounted on the catheter 910 is disposed at the position which corresponds to the center hole formed in the annular rubber elastic body 940 and the through hole 921 of the transdermal terminal 920.

In such a state, when the cap 960 is fastened by making the female threads 961 of the cap 960 threadedly engage with the male threads 923 of the transdermal terminal 920, the annular rubber elastic body 940 is pressed by the cap 960. When the annular rubber elastic body 940 is pressed by the cap 960, the annular rubber elastic body 940 applies a pressing force to the catheter 910 in a radial direction due to the tapered hole 963 formed in the cap 960. Accordingly, the catheter 910 is fixed to the transdermal terminal 920. Although a pressing force in the radial direction is applied to the catheter 910, the hard tube 950 is annularly mounted on the catheter 910 and hence, the deformation of the catheter 910 by a pressing force in the radial direction can be prevented.

When the transdermal catheter retaining set 900 having such a configuration is mounted on a transdermal part, that is, a skin 970 of a patient, the transdermal catheter retaining set 900 is brought into a state where a lower half of the transdermal terminal 920 including a lower flange 924 is embedded in the skin 970.

### CITATION LIST

### Patent Literature

PTL 1: JP 3-254758 A

### SUMMARY OF INVENTION

### Technical Problem

In the transdermal catheter retaining set 900 described in the patent literature 1, the cap 960 is fastened by making the female threads 961 formed on an inner peripheral surface of the cap 960 threadedly engage with the male threads 923 formed on an outer peripheral surface of the transdermal terminal 920. Accordingly, a fastening operation of the cap 960 is restricted by an upper end portion 920a (a distal end portion of the male threads 923) of the transdermal terminal 920. Accordingly, in performing fastening of the cap 960, when the inner wall surface 962a of a head portion 962 of the cap 960 comes into contact with an upper end portion 920a of the transdermal terminal 920, a further fastening operation is restricted. Accordingly, at this point of time, a pressing force applied to the annular rubber elastic body 940 is also restricted.

That is, a pressing force applied to the annular rubber elastic body 940 is restricted at the point of time that the inner wall surface 962a of the head portion 962 of the cap 960 comes into contact with the upper end portion 920a of the transdermal terminal 920. Accordingly, in the transdermal catheter retaining set 900 described in patent literature 1, there may be a case where a sufficient pressing force cannot be applied to the annular rubber elastic body 940. When a sufficient pressing force cannot be applied to the annular rubber elastic body 940, the annular rubber elastic body 940 cannot be brought into close contact with the whole recessed portion 922 and hence, there is a drawback that liquid sealing property between the outside of a body and the inside of the body becomes insufficient.

Further, in the transdermal catheter retaining set 900 described in patent literature 1, the case is assumed where a medical tube is a catheter, and the catheter is fixed to the skin 970. However, when a medical tube which differs from a catheter is fixed to the skin 970, there may be a case where the transdermal catheter retaining set 900 described in patent literature 1 is not applicable.

For example, the description will be made by taking a case of a ventricular assist system. In the ventricular assist system, a cable (also referred to as a drive line) which is disposed between a blood pump embedded in the inside of a body and a controller which controls the blood pump outside the body corresponds to a medical tube. In the inside of the drive line, an electric signal line for controlling the blood pump is incorporated. Further, in the case of ventricular assist system where a purge liquid having functions such as lubrication in the blood pump, cooling and the maintenance of sealing property is circulated, a purge liquid circulating pipe is also incorporated in the drive line besides the electric signal line.

It is often the case that such a ventricular assist system is used for a long period, and during a period that the ventricular assist system is used, a control of the blood pump by the controller cannot be stopped. Accordingly, in the case where a part which deteriorates due to the use of the part for a long period is provided in a medical tube securing jig for securing a drive line which forms a medical tube to a skin 970, a technique is requested which enables an exchange of a deteriorated part without stopping a control of the blood pump by the controller.

As a part which deteriorates due to the use of the part for a long period, a sealing member is exemplified. In the transdermal catheter retaining set 900 described in the above-mentioned patent literature 1, the sealing member corresponds to the annular rubber elastic body 940 (see FIG. 16). Accordingly, assuming the case where the transdermal catheter retaining set 900 is used as a medical tube securing jig for securing a drive line to a transdermal part in the ventricular assist system, to exchange the sealing member which corresponds to the annular rubber elastic body 940, it is necessary to perform an operation where the drive line which forms the medical tube is removed from the controller and, then, the sealing member which corresponds to the annular rubber elastic body 940 is made to slide on an outer peripheral surface of the drive line thus removing the sealing member from an end portion of the drive line.

However, as described previously, in the ventricular assist system, a control of the blood pump by the controller cannot be stopped and hence, the drive line cannot be removed from the controller. Accordingly, in the transdermal catheter retaining set 900 described in the patent literature 1, when the transdermal catheter retaining set 900 described in patent 1 is applied to the ventricular assist system, there also exists a drawback that an exchange of the sealing member which corresponds to the annular rubber elastic body 940 becomes difficult.

The present invention has been made in view of the above-mentioned circumstances, and it is an object of the present invention to provide a medical tube securing jig which exhibits excellent liquid sealing property between the outside of the body and the inside of the body and where a sealing member is exchangeable.

### Solution to Problem

[1] A medical tube securing jig according to the present invention is a medical tube securing jig for securing a medical tube to a transdermal part in inserting the medical tube from an outside of a body to an inside of the body, the medical tube securing jig including: a skin securing member having a flange portion which is embedded in the transdermal part and a cylindrical protruding body which protrudes from the flange portion at least toward the outside of the body; and a medical tube holding mechanism part which is mounted in a state where the medical tube holding mechanism part is inserted into the protruding body of the skin securing member so as to hold the medical tube, wherein a recessed portion whose opening is directed toward the outside of the body is formed on the protruding body of the skin securing member, a first through hole which allows the medical tube to pass therethrough is formed in a bottom surface of the recessed portion in a state where the first through hole penetrates the bottom surface toward the inside of the body and female threads are formed on a side wall surface of the recessed portion between the opening and the bottom surface, the medical tube holding mechanism part includes: a sealing member made of a material which has elasticity and liquid sealing property, the sealing member having a second through hole which allows the medical tube to pass therethrough, the sealing member being engageable with the recessed portion by fitting engagement in a state where the medical tube is made to pass through the second through hole, the sealing member being elastically deformed when a pressing force in a direction from the outside of the body to the inside of the body is applied to the sealing member in a state where the sealing member engages with the recessed portion by fitting engagement thus being brought into close contact with a side wall surface of the recessed portion, the bottom surface of the recessed portion, and the medical tube; and a sealing member pressing body disposed at a position more outside of the body than the sealing member, the sealing member pressing body having male threads being threadedly engageable with the female threads formed on the recessed portion, the sealing member pressing body having a third through hole which allows the medical tube to pass therethrough along a center axis of the male threads, the sealing member pressing body being configured to apply the pressing force to the sealing member in fastening the sealing member pressing body by making the male threads threadedly engage with the female threads in a state where the medical tube is made to pass through the third through hole, wherein a slit portion is formed in the sealing member, the slit portion reaching the other end side from one end side of the sealing member along a longitudinal direction of the medical tube, the slit portion reaching the second through hole in a radial direction.

According to the medical tube of the present invention, the mounting structure for mounting the sealing member pressing body on the skin securing member adopts a structure where the sealing member pressing body is mounted on the skin securing member by making the male threads of the sealing member pressing body threadedly engage with the female threads formed on the recessed portion of the skin securing member thus fastening the sealing member pressing body.

With such a configuration, it is possible to eliminate the occurrence of the case where, as in the case of the transdermal catheter retaining set 900 described in patent literature 1, the inner wall surface 962a of the head portion 962 of the cap 960 is brought into contact with the upper end portion 920a of the transdermal terminal 920 so that a further fastening operation is restricted and, at this point of time, a pressing force applied to the annular rubber elastic body 940 (sealing member) of the cap 960 (sealing member pressing body) is also restricted. Accordingly, the restriction imposed on a pressing force applied to the sealing member can be made as small as possible.

Accordingly, it is possible to apply a sufficient pressing force to the sealing member and hence, the sealing member can be elastically deformed without forming a gap in the recessed portion of the skin securing member. As a result, the sealing member can be brought into close contact with the side wall surface of the recessed portion, the bottom surface of the recessed portion, and the medical tube without forming a gap. In this case, the sealing member is brought into close contact with the side wall surface of the recessed portion, the bottom surface of the recessed portion, and an outer peripheral surface of the medical tube in face contact respectively. In this manner, the sealing member is brought into close contact with the side surface of the recessed portion, the bottom surface of the recessed portion, and the outer peripheral surface of the medical tube in face contact respectively and hence, the medical tube securing jig can have high liquid sealing property between the outside of the body and the inside of the body.

Further, the slit portion is formed on the sealing member and hence, the sealing member is exchangeable. Accordingly, for example, even in the case where the sealing member is deteriorated so that an exchange of the sealing member becomes necessary, the sealing member can be exchanged.

In this manner, according to the medical tube securing jig of the present invention, it is possible to provide a medical tube securing jig which exhibits excellent liquid sealing property between the outside of the body and the inside of the body, and enables an exchange of the sealing member.

In the medical tube securing jig according to the present invention, the sealing member is disposed at a position closest to the inside of the body in the medical tube holding mechanism part of the medical tube securing jig. Accordingly, the part which adheres to a hypodermal tissue which is a living tissue is only the sealing member besides the skin securing member. Further, the sealing member is elastically deformed by being pressed by the sealing member pressing body so that the sealing member is brought into a state where the sealing member is embedded in the recessed portion. Accordingly, the sealing member is brought into close contact with the side wall surface of the recessed portion, the bottom surface of the recessed portion, and the outer peripheral surface of the medical tube in face contact and hence, the portion which is brought into contact with a living tissue is only a very small portion of the sealing member. Accordingly, an adhesion range of the sealing member can be suppressed to a minimum range and hence, the sealing member can be easily exchanged even when it is necessary to exchange the sealing member.

In the medical tube securing jig of the present invention, the sealing member plays the role of gripping a medical tube such that the medical tube does not move in the transdermal part besides the role of providing sealing between the outside of the body and the inside of the body with high liquid sealing property. Accordingly, it is sufficient to provide only the sealing member and the sealing member pressing body which presses the sealing member as the main parts which form the medical tube holding mechanism part of the medical tube securing jig. Accordingly, the number of parts can be reduced thus giving rise to an advantageous effect that the structure of the medical tube securing jig can be simplified.
[2] In the medical tube securing jig according to the present invention, it is preferable that the medical tube holding mechanism part have a washer interposed between the sealing member and the sealing member pressing body, and the washer be made of a material where a frictional force between the washer and the sealing member pressing body is smaller than a frictional force between the washer and the sealing member.

By interposing this washer between the sealing member and the sealing member pressing body, in performing a fastening operation of the sealing member pressing body, it is possible to prevent a rotational force of the sealing member pressing body from being transmitted to the sealing member. Accordingly, in performing the fastening operation of the sealing member pressing body, it is possible to prevent so-called “co-rotation” that the sealing member is rotated together with the rotation of the sealing member pressing body.
[3] In the medical tube securing jig according to the present invention, it is preferable that the skin securing member further have a brim portion which is disposed at a position on a more outside of the body than the flange portion and spaced apart from the flange portion, and the brim portion be disposed such that a surface of the brim portion directed toward the inside of the body is in contact with a surface of the transdermal part on the outside of the body in a state where the skin securing member is fixed to the transdermal part.

With the provision of such a brim portion, when the skin securing member is fixed to a skin, the skin securing member can be fixed to the skin in a stable state.
[4] In the medical tube securing jig according to the present invention, it is preferable that the flange portion be made of porous metal having biocompatibility.

In this manner, the flange portion is made of porous metal having biocompatibility and hence, a tissue of a skin and the flange portion easily adhere to each other, and the invasion of germs or the like into a living body can be prevented whereby it is possible to increase an effect of suppressing the occurrence of infectious diseases. For example, titanium in a sponge form can be used as porous metal. However, the porous metal is not limited to such titanium.
[5] In the medical tube securing jig according to the present invention, it is preferable that the flange portion made of porous metal and the protruding body be formed as separate parts from each other, and the protruding body be disposed so as to protrude not only toward the outside of the body but also toward the inside of the body, assuming a portion of the protruding body protruding toward the inside of the body is a body inside protruding body, a space portion which allows the body inside protruding body to pass therethrough be formed on a center portion of the flange portion made of the porous metal, and the flange portion made of the porous metal and the body inside protruding body be joined to each other in a state where the body inside protruding body is made to pass through 000the space portion.

The flange is made of porous metal and hence, the flange portion made of porous metal and the protruding body are formed as separate parts from each other. Accordingly, it is possible to provide the skin securing member where the flange portion is made of porous metal by forming the space portion which allows the body inside protruding body to pass therethrough in the center portion of the flange portion made of porous metal and by joining the flange portion made of porous metal and the body inside protruding body to each other in a state where the body inside protruding body is made to pass through the space portion.
[6] In the medical tube securing jig according to the present invention, it is preferable that the flange portion be made of a non-woven fabric having biocompatibility.

In this manner, the flange portion is made of a non-woven fabric having biocompatibility and hence, a tissue of a skin and the flange portion 110A easily adhere to each other, the invasion of germs or the like into a living body can be prevented whereby it is possible to increase an effect of suppressing the occurrence of infectious diseases. Further, the flange portion is made of a soft material and hence, it is also possible to acquire an advantageous effect that a burden imposed on a living body can be reduced when the flange portion is embedded in a skin.
[7] In the medical tube securing jig according to the present invention, it is preferable that the flange portion which is made of the non-woven fabric and the protruding body be formed as separate parts from each other, and the protruding body be disposed so as to protrude not only toward the outside of the body but also toward the inside of the body, and assuming a portion of the protruding body protruding toward the inside of the body is a body inside protruding body, a space portion which allows the body inside protruding body to pass therethrough be formed on a center portion of the flange portion made of the non-woven fabric, and the flange portion made of the non-woven fabric and the body inside protruding body be joined to each other in a state where the body inside protruding body is made to pass through the space portion.

In this manner, the flange is made of a non-woven fabric and hence, the flange portion made of a non-woven fabric and the protruding body are formed as separate parts from each other. Accordingly, it is possible to provide the skin securing member where the flange portion is made of a non-woven fabric by forming the space portion which allows the body inside protruding body to pass therethrough in the center portion of the flange portion made of a non-woven fabric and by joining the flange portion made of a non-woven fabric and the body inside protruding body to each other in a state where the body inside protruding body is made to pass through the space portion.
[8] In the medical tube securing jig according to the present invention, it is preferable that polytetrafluoroethylene (PTFE) felt be used as the non-woven fabric.

The PTFE felt is a material having excellent corrosion resistance and high compatibility with a living tissue and hence, the PTFE felt is suitable as a member to be embedded in a living body, and exhibits excellent adhesiveness with a tissue of a skin.
[9] In the medical tube securing jig according to the present invention, it is preferable that the medical tube securing jig further include a waterproof cap which is mounted on an end portion of the sealing member pressing body on the outside of the body in addition to the skin securing member and the medical tube holding mechanism part, wherein the waterproof cap be made of a material having elasticity and liquid sealing property, the waterproof cap having a gap insertion portion which is insertable into a gap formed between the sealing member pressing body and the medical tube so as to fill the gap, and the waterproof cap being annularly mounted on the medical tube such that the waterproof cap is slidable along a longitudinal direction of the medical tube in a state where the waterproof cap is brought into close contact with an outer peripheral surface of the medical tube.

With the provision of such a waterproof cap, the medical tube securing jig according to the present invention can satisfy a waterproof specification on the outside of the body and hence, it is possible to prevent the intrusion of the liquid such as a water into the medical tube holding mechanism part. It is also possible to prevent the intrusion of dust or the like into the medical tube holding mechanism part in addition to the prevention of the intrusion of the liquid.
[10] In the medical tube securing jig according to the present invention, it is preferable that the medical tube be a cable which connects a blood pump disposed in the inside of the body and a controller disposed on the outside of the body in a ventricular assist system.

In this manner, in the medical tube securing jig according to the present invention, the medical tube is a cable (drive line) which connects the blood pump disposed in the inside of the body and the controller disposed on the outside of the body to each other in the ventricular assist system. With such a configuration, the medical tube securing jig according to the present invention can acquire a particularly large advantageous effect. That is, a medical tube securing jig used in a ventricular assist system is required to satisfy, not to mention a condition that the medical tube securing jig exhibits excellent liquid sealing property between the outside of the body and the inside of the body, a condition that a sealing member which is a member deteriorated with time is exchangeable without stopping a control of a blood pump by a controller since the control of the blood pump by the controller cannot be stopped during the use of the ventricular assist system. By taking into account these points, according to the medical tube securing jig of the present invention, not to mention that the medical tube securing jig of the present invention exhibits excellent liquid sealing property between the outside of the body and the inside of the body, the sealing part can be exchanged without stopping a control of the blood pump by the controller even during the use of the ventricular assist system since the slit portion is formed in the sealing part.

### BRIEF DESCRIPTION OF DRAWINGS

FIG. 1A to FIG. 1C are views for describing a medical tube securing jig 1 according to an embodiment 1.

FIG. 2 is a view for describing one example of a ventricular assist system.

FIG. 3 is a view for describing constitutional parts of the medical tube securing jig 1 according to the embodiment 1 as individual parts.

FIG. 4 is a flowchart describing assembling steps of the medical tube securing jig 1 according to the embodiment 1.

FIG. 5A and FIG. 5B are views showing the medical tube securing jig 1 after the medical tube securing jig 1 is assembled in accordance with the respective steps shown in FIG. 4.

FIG. 6 is a cross-sectional view of a main part showing a state where the medical tube securing jig 1 according to the embodiment 1 is mounted on a skin 500.

FIG. 7 is a view for describing constitutional parts of a medical tube securing jig 1A according to an embodiment 2 as individual parts.

FIG. 8 is a view showing a skin securing member 100A in a state where a flange portion 110A and a body inside protruding body 120a are joined to each other.

FIG. 9A and FIG. 9B are views showing the medical tube securing jig 1A according to the embodiment 2 after the medical tube securing jig 1A is assembled in accordance with the respective steps shown in FIG. 4.

FIG. 10 is a cross-sectional view of a main part showing a state where the medical tube securing jig 1A according to the embodiment 2 is fixed to a transdermal part, that is, a skin 500.

FIG. 11 is a view for describing constitutional parts of a medical tube securing jig 1B according to an embodiment 3 as individual parts.

FIG. 12 is a plan view of a protruding body 120 of the medical tube securing jig 1B according to the embodiment 3 in the form that the protruding body 120 is taken out from the medical tube securing jig 1B.

FIG. 13 is a view of a skin securing member 100B in a state where a flange portion 110B and the body inside protruding body 120a are joined to each other.

FIG. 14A and FIG. 14B are views showing the medical tube securing jig 1B according to the embodiment 3 after the medical tube securing jig 1B is assembled in accordance with the respective steps shown in FIG. 4.

FIG. 15 is a cross-sectional view of a main part showing a state where the medical tube securing jig 1B according to the embodiment 3 is fixed to a transdermal part, that is, a skin 500.

FIG. 16 is a cross-sectional view for describing a transdermal catheter retaining set 900 described in patent literature 1.

### DESCRIPTION OF EMBODIMENTS

Hereinafter, embodiments of the present invention are described.

### Embodiment 1

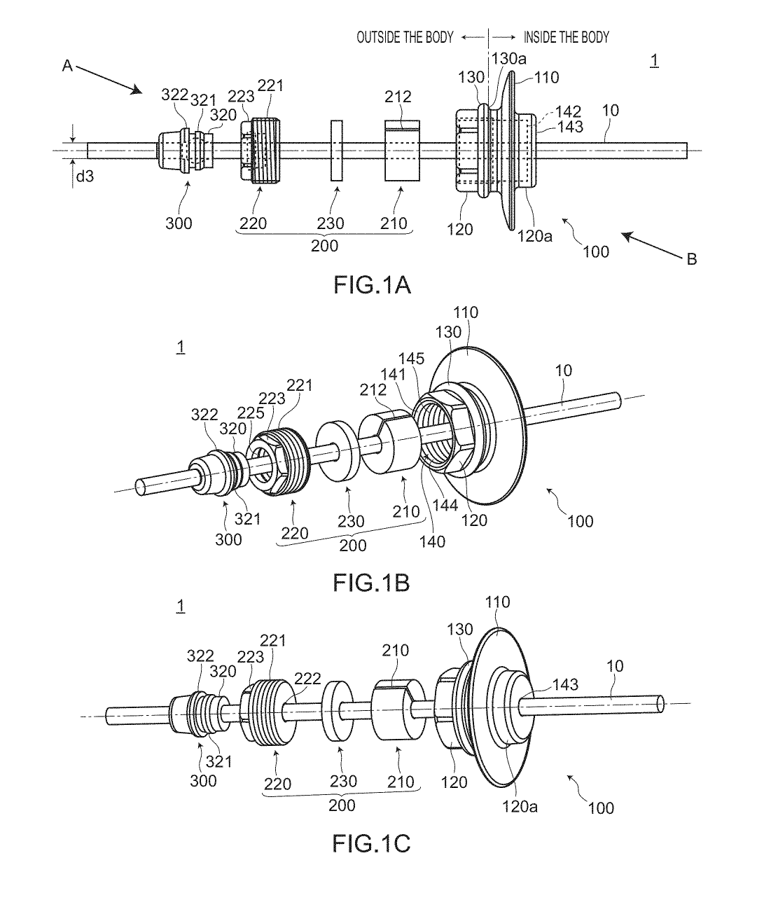

FIG. 1A to FIG. 1C are views for describing a medical tube securing jig 1 according to the embodiment 1. FIG. 1A to FIG. 1C shows a state before the medical tube securing jig 1 according to the embodiment 1 is assembled. FIG. 1A is a plan view, FIG. 1B is a perspective view as viewed from a direction indicated by an arrow A in FIG. 1A, and FIG. 1C is a perspective view as viewed from a direction indicated by an arrow B in FIG. 1A. Hereinafter, the medical tube securing jig 1 according to the embodiment 1 is described with reference to FIG. 1A to FIG. 1C.

The medical tube securing jig 1 according to the embodiment 1 includes: a skin securing member 100 fixed to a skin; a medical tube holding mechanism part 200 mounted in a state where the medical tube holding mechanism part 200 is inserted into a protruding body 120 of the skin securing member 100 and provided for holding a medical tube 10; and a waterproof cap 300 provided for applying a waterproof specification to the medical tube securing jig 1 according to the embodiment 1 outside a body by being mounted on the medical tube holding mechanism part 200. The skin securing member 100, the medical tube holding mechanism part 200, and the waterproof cap 300 are described in detail later.

In the medical tube securing jig 1 according to the embodiment 1, assume that a medical tube 10 which forms an object to be fixed is, for example, a cable which connects a blood pump disposed inside the body and a controller disposed outside the body in a ventricular assist system. The cable is also referred to as a drive line. In the description made hereinafter, the drive line is expressed as “medical tube 10”.

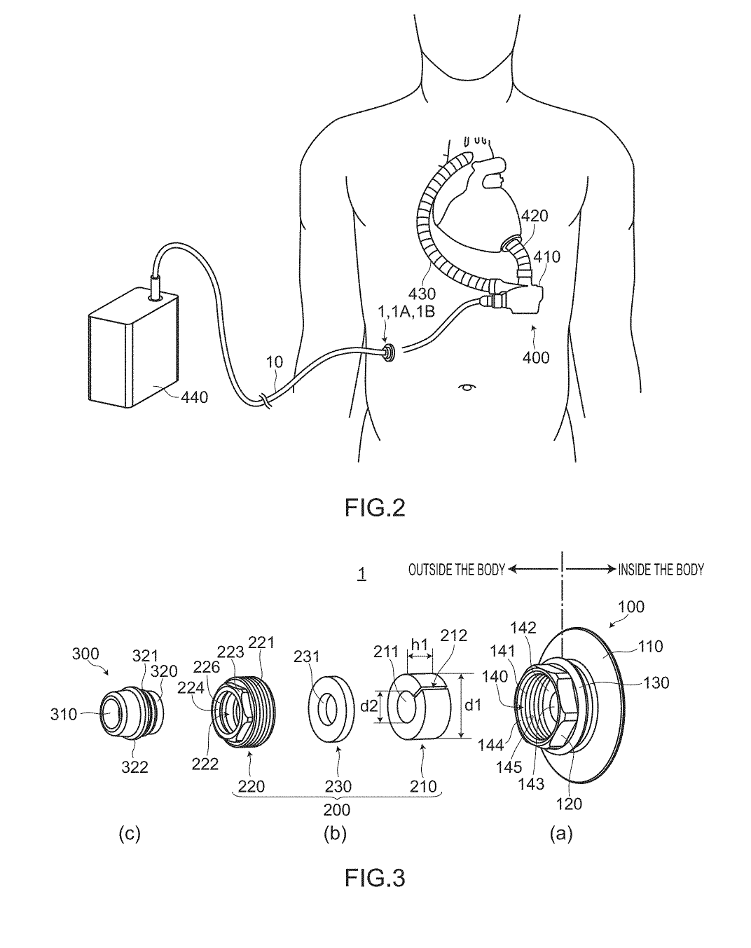

FIG. 2 is a view for describing one example of the ventricular assist system 400. As shown in FIG. 2, the ventricular assist system 400 has: a blood pump 410 embedded inside the body; artificial blood vessels 420, 430 provided for connecting the blood pump 410 and a blood flow in a heart; a controller 440 having a function of controlling the blood pump 410 outside the body; the medical tube 10 disposed between the blood pump 410 and the controller 440 as a drive line; and the medical tube securing jig 1 provided for securing the medical tube 10 to a transdermal part. The medical tube securing jig 1 is expressed as medical tube securing jigs 1A, 1B in an embodiment 2 and an embodiment 3 described later.

In FIG. 2, the medical tube securing jig 1 according to the embodiment 1 and the medical tube securing jigs 1A, 1B according to the embodiments 2, 3 described later are described in a simplified manner. An electric signal line (not shown in the drawing) is incorporated in the medical tube 10. When the ventricular assist system 400 is a ventricular assist system where a purge liquid having functions such as lubrication, cooling, and maintenance of sealing property in the blood pump 410 is circulated, a purge liquid circulating pipe (not shown in the drawing) is also incorporated in the medical tube 10 besides the electric signal line.

In this embodiment, assume that an outer diameter of the medical tube 10 is approximately 8 mm. However, the outer diameter of the medical tube 10 is not limited to 8 mm. Further, although it is not explicitly described in FIG. 1A to FIG. 1C, with respect to the medical tube 10 disposed between the controller 440 and the blood pump 410, a tissue substituting artificial fiber fabric which is also referred to as a fabric covers an outer peripheral surface of the medical tube 10 at least between an area in the vicinity of an inlet to a living body (medical tube securing jig 1) and the blood pump 410.

FIG. 3 is a view for describing constitutional parts of the medical tube securing jig 1 according to the embodiment 1 as individual parts. FIG. 3 is a perspective view of the constitutional parts of the medical tube securing jig 1 shown in FIG. 1A to FIG. 1C in the form that the constitutional parts are taken out from the medical tube 10. In FIG. 3, a portion expressed by (a) indicates a skin securing member 100, a portion expressed by (b) indicates constitutional parts (a sealing member 210, a washer 230, and a sealing member pressing body 220) of the medical tube holding mechanism part 200, and a portion expressed by (c) indicates the waterproof cap 300.

Hereinafter, the skin securing member 100, the medical tube holding mechanism part 200, and the waterproof cap 300 are sequentially described in detail with reference to FIG. 3 and FIG. 1A to FIG. 1C described previously.

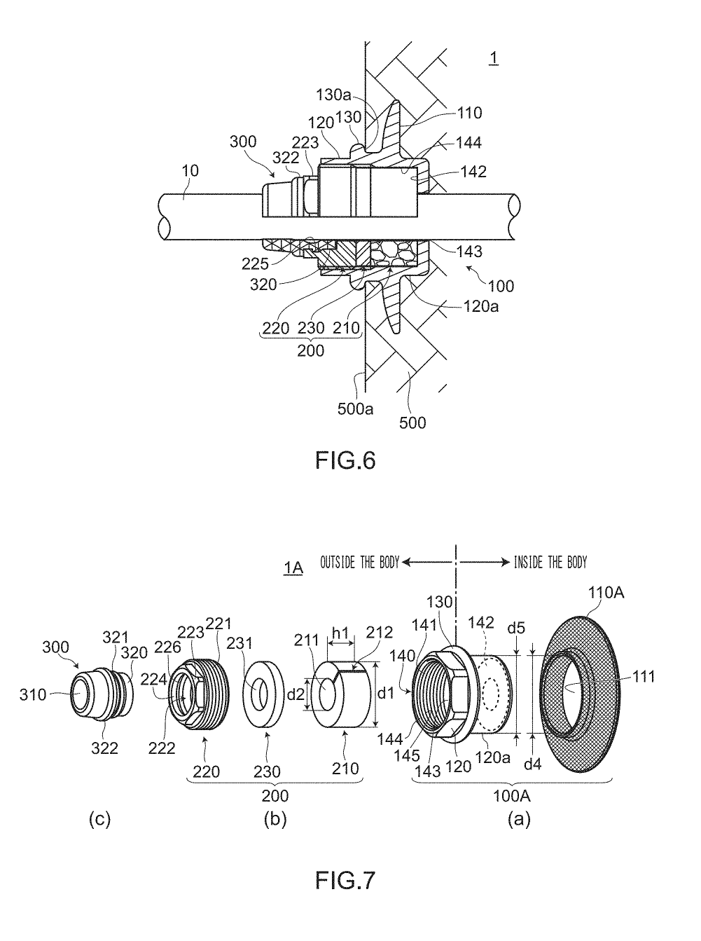

First, the skin securing member 100 is described. As shown in FIG. 1A to FIG. 1C and FIG. 3, as the external appearance configuration, the skin securing member 100 has: a flange portion 110 embedded in a skin 500 (see FIG. 6); a cylindrical protruding body 120 disposed in a protruding manner from the flange portion 110 toward at least the outside of the body; and a brim portion 130 disposed at a position on the outside of the body compared to the flange portion 110 and spaced apart from the flange portion 110.

In the medical tube securing jig 1 according to the embodiment 1, the protruding body 120 is disposed such that the producing body 120 protrudes not only toward the outside of the body but also toward the inside of the body (see FIG. 1A and FIG. 1C). In this embodiment 1, a portion of the protruding body 120 which protrudes toward the inside of the body is referred to as “body inside protruding body 120a”. The brim portion 130 is disposed such that a surface 130a (see FIG. 1A) of the brim portion 130 which is directed toward the inside of the body is brought into contact with a surface 500a of the skin 500 (see FIG. 6) when the skin securing member 100 is fixed to the skin 500 (see FIG. 6). Such configuration is adopted substantially in the same manner by the medical tube securing jigs 1A, 1B according to the embodiments 2 and 3 described later.

A recessed portion 140 whose opening 141 is directed toward the outside of the body is formed in the protruding body 120 of the skin securing member 100. A through hole (referred to as a first through hole) 143 which allows the medical tube 10 to pass therethrough is formed in a bottom surface 142 of the recessed portion 140 (see a portion expressed by (a) in FIG. 3) such that the through hole 143 penetrates the bottom surface 142 toward the inside of the body. Female threads 145 are formed on a side wall surface 144 between the opening 141 and the bottom surface 142 of the recessed portion 140. The bottom surface 142 of the recessed portion 140 is formed in the body inside protruding body 120a of the protruding body 120.

The skin securing member 100 having such a configuration is fixed to a skin which is a living tissue and hence, it is preferable that the skin securing member 100 be made of metal which exhibits excellent corrosion resistance and high compatibility with a living tissue. As metal which has excellent corrosion resistance and high compatibility with a living tissue, for example, titanium, a titanium alloy or the like can be exemplified.

Next, the medical tube holding mechanism part 200 is described. As shown in FIG. 1A to FIG. 1C and the portion expressed by (b) in FIG. 3, the medical tube holding mechanism part 200 has: the sealing member 210; the sealing member pressing body 220 which applies a pressing force to the sealing member 210; and the washer 230 interposed between the sealing member 210 and the sealing member pressing body 220.

The sealing member 210 is made of a material which is elastically deformable when a pressing force is applied to the sealing member 210. As indicated by the portion expressed by (b) in FIG. 3, the sealing member 210 has a circular columnar shape where a height h1 is smaller than an outer diameter d1. Further, the sealing member 210 has a through hole (referred to as a second through hole) 211 which allows the medical tube 10 to pass therethrough. A diameter d2 of the second through hole 211 and an outer diameter d3 of the medical tube 10 (see FIG. 1A) are substantially equal to each other.

“slit portion 212” is formed in the sealing member 210. The slit portion 212 is formed such that the slit portion 212 extends from one end side of the sealing member 210 and reaches the other end side of the sealing member 210 along a longitudinal direction of the medical tube 10 with respect to a height h1 direction of the sealing member 210, and reaches the second through hole 211 with respect to a radial direction of the sealing member 210.

The sealing member 210 having such a configuration is configured to be engageable with the recessed portion 140 of the skin securing member 100 by fitting engagement in a state where the medical tube 10 is made to pass through the second through hole 211. In a state where the sealing member 210 is fitted in the recessed portion 140, when a pressing force in a direction directed from the outside of the body to the inside of the body is applied to the sealing member 210, the sealing member 210 is elastically deformed and is brought into close contact with the side wall surface 144 of the recessed portion 140, the bottom surface 142 of the recessed portion 140, and an outer peripheral surface of the medical tube 10 whereby the outside of the body and the inside of the body are brought into a liquid sealing state using the sealing member 210 as a boundary.

“slit portion 212” is formed in the sealing member 210. Accordingly, in mounting the sealing member 210 on the medical tube 10, the sealing member 210 can be mounted on the medical tube 10 by expanding the slit portion 212 and by pushing the sealing member 210 from an outer peripheral surface side of the medical tube 10. On the other hand, in removing the sealing member 210 from the medical tube 10, the slit portion 212 is expanded, and the sealing member 210 can be removed from the outer peripheral surface of the medical tube 10.

Accordingly, in mounting the sealing member 210 on the medical tube 10 or in removing the sealing member 210 from the medical tube 10, it is unnecessary to perform a mounting operation where an end portion of the medical tube 10 is made to pass through the through hole (second through hole) 211 of the sealing member 210, and the sealing member 210 is slid to a predetermined mounting position on the medical tube 10, and a removing operation where the sealing member 212 is slid from a mounting position of the sealing member 212 along the longitudinal direction of the medical tube 10 and the sealing member 212 is removed from the end portion of the medical tube 10. Accordingly, not to mention that mounting and removal of the sealing member 210 on and from the medical tube 10 are performed easily, it is unnecessary to remove the medical tube 10 from equipment such as a controller when the end portion of the medical tube 10 is connected to the equipment.

The sealing member pressing body 220 is provided for pressing and deforming the sealing member 210 by applying a pressing force to the sealing member 210. As shown in FIG. 1A to FIG. 1C and the portion expressed by (b) in FIG. 3, the sealing member pressing body 220 is disposed at a position more outside the body than the sealing member 210. The sealing member pressing body 220 has male threads 221 which are threadedly engageable with female threads 145 formed on the recessed portion 140 of the skin securing member 100. The sealing member pressing body 220 also has a through hole (referred to as a third through hole) which allows the medical tube 10 to pass therethrough along a center axis of the male threads 221. The sealing member pressing body 220 also has a screw head portion 223 for applying a fastening force at the time of fastening the sealing member pressing body 220 to the recessed portion 140 by making the male threads 221 threadedly engage with the female threads 145 of the recessed portion 140. The screw head portion 223 has a shape substantially equal to a shape of a hexagonal bolt, for example, so as to enable fastening using a fastening tool such as a wrench.

With respect to the sealing member pressing body 220 having such a configuration, when the sealing member pressing body 221 is fastened to the recessed portion 140 by making the male threads 221 threadedly engage with the female threads 145 of the recessed portion 140 in a state where the medical tube 10 is made to pass through the third through hole 222, the sealing member pressing body 221 applies a pressing force to the sealing member 210 so that the sealing member 210 is elastically deformed.

In the sealing member pressing body 220, a recessed portion 224 having a larger diameter than the third through hole 222 is formed in the screw head portion 223. The above-mentioned third through hole 222 is formed directed in a direction from the bottom surface of the recessed portion 224 toward the inside of the body. The recessed portion 224 formed in the screw head portion 223 has a larger diameter than the third through hole 222 and hence, in a state where the medical tube 10 is made to pass through the third through hole 222, a gap 225 (see FIG. 1B) is formed between the recessed portion 224 and the medical tube 10 in the screw head portion 223. The gap 225 is provided for inserting the waterproof cap 300. Accordingly, the gap 225 is expressed as a waterproof cap inserting gap 225.

A recessed groove 226 (see the portion expressed by (b) in FIG. 3) is formed on an inner wall surface of the waterproof cap inserting gap 225 over the entire circumference in a circumferential direction. The recessed groove 226 is formed for making a projecting portion 321 (described later) formed on the waterproof cap 300 engage with the recessed groove 226 by fitting engagement when the waterproof cap 300 is inserted into the waterproof cap inserting gap 225.

Although the sealing member pressing body 220 is not directly brought into contact with a living tissue unlike the skin securing member 100, it is preferable that the sealing member pressing body 220 be made of metal (for example, titanium, a titanium alloy or the like) having excellent corrosion resistance and having high compatibility with a living tissue in the same manner as the skin securing member 100. However, the sealing member pressing body 220 may be made of stainless steel or the like.

The washer 230 has a through hole (fourth through hole) 231 through which the medical tube 10 pass. It is preferable that the fourth through hole 231 have a diameter which allows the formation of a slight gap between the medical tube 10 and the fourth through hole 231 when the medical tube 10 is made to pass through the fourth through hole 231. The washer 230 is made of a material by which a frictional force between the washer 230 and the sealing member pressing body 220 is smaller than a frictional force between the washer 230 and the sealing member 210. For this end, it is preferable to use metal as the material of the washer 230. For example, metal substantially equal to metal for forming the sealing member pressing body 220 can be used as the material of the washer 230.

By interposing such a washer 230 between the sealing member 210 and the sealing member pressing body 220, it is possible to prevent a rotational force of the sealing member pressing body 220 from being transmitted to the sealing member 210 when a fastening operation of the sealing member pressing body 220 is performed.

That is, assume a case where a fastening operation of the sealing member pressing body 220 is performed without interposing the washer 230 between the sealing member 210 and the sealing member pressing body 220. In this case, a rotational force of the sealing member pressing body 220 is transmitted to the sealing member 210 and hence, a phenomenon that the sealing member 210 is rotated together with the rotation of the sealing member pressing body 220, that is, so-called “co-rotation” is generated.

When “co-rotation” of the sealing member 210 is generated, the medical tube 10 which is in a close contact state with the sealing member 210 is also rotated. When the medical tube 10 is rotated, the following drawbacks occur. That is, a patient feels a pain or a connection state between the medical tube 10 and equipment connected to the medical tube (for example, a blood pump of a ventricular assist system, a controller which controls the blood pump or the like) is adversely affected. By interposing the washer 230 made of metal between the sealing member 210 and the sealing member pressing body 220, the occurrence of such drawbacks can be overcome.

Next, the waterproof cap 300 is described. The waterproof cap 300 is made of a material having elasticity and liquid sealing property such as silicone. As shown in FIG. 1A to FIG. 1C and the portion expressed by (c) in FIG. 3, the waterproof cap 300 is annularly mounted on the medical tube 10 such that the waterproof cap 300 is slidable on an outer peripheral surface of the medical tube 10 along the longitudinal direction of the medical tube 10.

The waterproof cap 300 has a through hole (fifth through hole) 310 which allows the medical tube 10 to pass therethrough. The fifth through hole 310 has an inner diameter substantially equal to an outer diameter d3 of the medical tube 10 (see FIG. 1A to FIG. 1C). The fifth through hole 310 is formed such that the waterproof cap 300 is brought into close contact with the medical tube 10 due to an elastic force in a state where the medical tube 10 is made to pass through the fifth through hole 310. However, even in a state where the waterproof cap 300 is brought into close contact with the medical tube 10 due to an elastic force, the waterproof cap 300 is allowed to slide along a longitudinal direction of the medical tube 10.

The waterproof cap 300 has a gap insertion portion 320 which is insertable into the waterproof cap inserting gap 225 formed between sealing member pressing body 220 and the medical tube 10 at the screw head portion 223 of the sealing member pressing body 220. With such a configuration, when the gap insertion portion 320 of the waterproof cap 300 is inserted into the waterproof cap inserting gap 225 formed in the screw head portion 223, a state is obtained where sealing property is provided between the screw head portion 223 and the medical tube 10. Accordingly, the medical tube securing jig 1 according to the embodiment 1 can satisfy a waterproof specification outside the body.

The projecting portion 321 is formed on an outer peripheral surface of the gap insertion portion 320 of the waterproof cap 300. The projecting portion 321 is integrally formed with the gap insertion portion 320 over the entire circumference in a circumferential direction. With such a configuration, when the gap insertion portion 320 is inserted into the waterproof cap inserting gap 225 formed in the screw head portion 223, the projecting portion 321 formed on the gap insertion portion 320 is fitted in the recessed groove 226 formed in the screw head portion 223.

Accordingly, the waterproof cap 300 can be mounted on the sealing member pressing body 220 with certainty and hence, it is possible to prevent the occurrence of a drawback that the waterproof cap 300 inadvertently falls unless the waterproof cap 300 is intentionally removed. Further, waterproof cap 300 is mounted on the sealing member pressing boy 220 with certainty and hence, it is also possible to prevent the occurrence of a drawback that fastening (fixing by thread engagement) of the sealing member pressing body 220 to the recessed portion 140 is loosened.

A brim portion 322 is integrally formed with the gap insertion portion 320 at a base portion of the gap insertion portion 320. The brim portion 322 is brought into a contact state with the screw head portion 223 in a state where the gap insertion portion 320 is inserted into the waterproof cap inserting gap 225. With such a configuration, sealing property can be provided to the waterproof cap inserting gap 225 formed in the screw head portion 223 with more certainty and hence, the medical tube securing jig 1 can acquire higher waterproof property.

Next, assembling steps of the medical tube securing jig 1 according to the embodiment 1 are described.

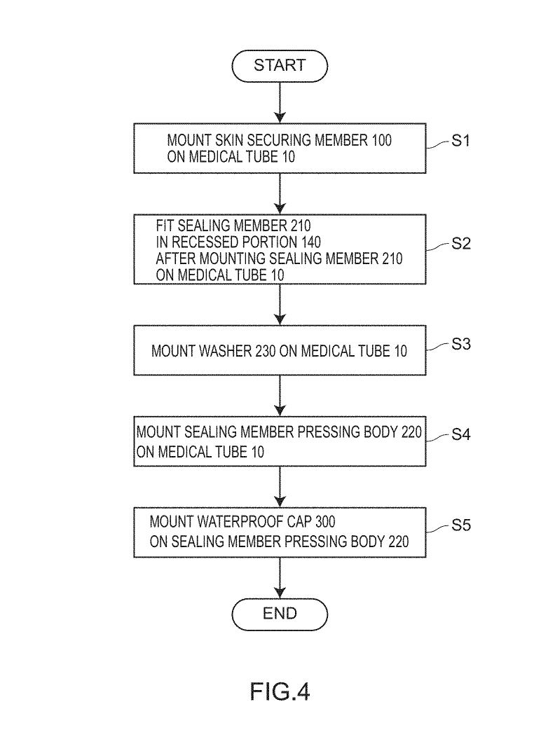

FIG. 4 is a flowchart describing the assembling steps of the medical tube securing jig 1 according to the embodiment 1. As shown in FIG. 4, first, the skin securing member 100 is mounted on the medical tube 10 (first step S1). To be more specific, the medical tube 10 is made to pass through the through hole (first through hole) 143 formed in the skin securing member 100 so as to obtain a state where the skin securing member 100 is annularly mounted on the medical tube 10.

Next, the sealing member 210 is mounted on the medical tube and, thereafter, the sealing member 210 is fitted in the recessed portion 140 of the skin securing member 100 (second step S2). To be more specific, in a state where the sealing member 210 is annularly mounted on the medical tube 10, the sealing member 210 is slid and is fitted in the recessed portion 140 of the skin securing member 100.

Next, the washer 230 is mounted on the medical tube 10 (third step S3). To be more specific, the medical tube 10 is made to pass through the through hole (fourth through hole) 231 formed in the washer 230 so as to obtain a state where the washer 230 is annularly mounted on the medical tube 10.

Then, the sealing member pressing body 220 is mounted on the medical tube 10 (fourth step S4). To be more specific, the medical tube 10 is made to pass through the through hole (third through hole) 222 formed in the sealing member pressing body 220 so as to obtain a state where the sealing member pressing body 220 is annularly mounted on the medical tube 10. In such a state, the male threads 221 formed on the sealing member pressing body 220 threadedly engage with the female threads 145 formed on the recessed portion 140 of the skin securing member 100, and the sealing member pressing body 220 is fastened to the recessed portion 140 by rotating the screw head portion 223 of the sealing member pressing body 220. At this stage of the operation, the washer 230 is interposed between the sealing member 210 and the sealing member pressing body 220. Accordingly, when the fastening of the sealing member pressing body 220 is performed, it is possible to prevent the occurrence of “co-rotation” where the sealing member 210 is rotated along with fastening of the sealing member pressing body 220.

Then, the waterproof cap 300 is mounted on the sealing member pressing body 220 (fifth step S5). To be more specific, the medical tube 10 is made to pass through the through hole (fifth through hole) 310 of the waterproof cap 300 so as to obtain a state where the waterproof cap 300 is annularly mounted on the medical tube 10. In this state, the waterproof cap 300 is slid so as to insert the gap insertion portion 320 into the waterproof cap inserting gap 225. In such an operation, the gap insertion portion 320 is inserted until the projecting portion 321 formed in the gap insertion portion 320 is fitted in the recessed groove 226 formed in the screw head portion 223. Accordingly, a state is obtained where the brim portion 322 which is integrally formed with the gap insertion portion 320 is brought into contact with the screw head portion 223.

The medical tube securing jig 1 according to the embodiment 1 can be assembled by performing the above-mentioned first to fifth steps.

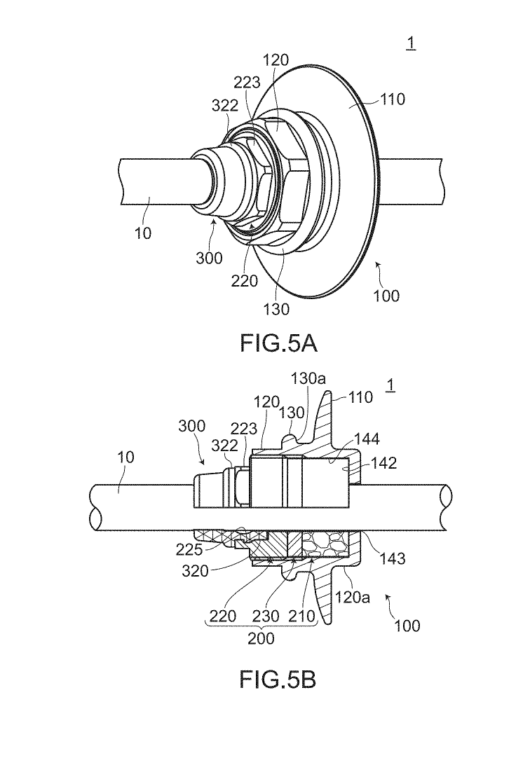

FIG. 5A and FIG. 5B are views showing the medical tube securing jig 1 after the medical tube securing jig 1 is assembled in accordance with the respective steps shown in FIG. 4. FIG. 5A is a perspective view, and FIG. 5B is a plan view with a main part shown in cross section. As shown in FIG. 5A and FIG. 5B, the sealing member 210 is brought into a state where the sealing member 210 is elastically deformed in the recessed portion 140 of the skin securing member 100 without forming a gap due to a pressing force of the sealing member pressing body 220. That is, the sealing member 210 is brought into a state where the sealing member 210 is in close contact with the side wall surface 144 of the recessed portion 140 and the bottom surface 142 of the recessed portion 140 without forming a gap, and is also in close contact with the medical tube 10 without forming a gap. Although the slit portion 212 is formed in the sealing member 210, it was confirmed that respective opposedly facing end portions of the sealing member 210 at the slit portion 212 were also brought into close contact state due to elastic deformation of the sealing member 210 without forming a gap in the recessed portion 140 due to a pressing force of the sealing member pressing body 220.

FIG. 6 is a cross-sectional view of a main part showing a state where the medical tube securing jig 1 according to the embodiment 1 is fixed to a transdermal part, that is, the skin 500. The state where the medical tube securing jig 1 is fixed to the skin 500 means a state where the skin securing member 100 is fixed to the skin 500 due to adhesion between the skin securing member 100 and a part of the skin 500 which is a living tissue.

As shown in FIG. 6, the skin securing member 100 of the medical tube securing jig 1 is fixed to the skin 500 so that a state is brought about where the medical tube 10 is fixed to the skin 500. That is, the medical tube 10 is held on the skin securing member 100 in a state where the movement of the medical tube 10 is prevented by constitutional parts (the sealing member 210, the washer 230, and the sealing member pressing body 220) of the medical tube holding mechanism part 200. Accordingly, a state is brought about where the medical tube 10 is fixed to the skin 500.

With respect to the medical tube securing jig 1 according to the embodiment 1, the case is exemplified where the medical tube securing jig 1 is used in the ventricular assist system. Accordingly, as shown in FIG. 2, out of both end portions of the medical tube 10, the end portion at the body side with respect to the medical tube securing jig 1 is connected to the blood pump 410, and the end portion disposed outside the body with respect to the medical tube securing jig 1 is connected to the controller 440 which controls the blood pump 410.

In a state where the medical tube securing jig 1 according to the embodiment 1 is fixed to the skin 500 as shown in FIG. 6, as described with reference to the FIG. 5A and FIG. 5B, a state is brought about where the sealing member 210 is elastically deformed by being pressed by the sealing member pressing body 220 and fills the recessed portion 140 of the skin securing member 100. Accordingly, the sealing member 210 is brought into close contact with the side wall surface 144 of the recessed portion 140, the bottom surface 142 of the recessed portion 140 and the outer peripheral surface of the medical tube 10 respectively in face contact. In this manner, the sealing member 210 is brought into close contact with the side wall surface 144 of the recessed portion 140, the bottom surface 142 of the recessed portion 140, and the outer peripheral surface of the medical tube 10 in face contact and hence, the medical tube securing jig 1 has high liquid sealing property at the outside of the body and the inside of the body. Accordingly, it is possible to prevent the invasion of germs or the like into the inside of the body with certainty.

The sealing member 210 is brought into close contact with the side wall surface 144 of the recessed portion 140, the bottom surface 142 of the recessed portion 140, and the outer peripheral surface of the medical tube 10 respectively in face contact. Accordingly, at the skin securing member 100, “displacement” of the medical tube 10 along an axial direction of the medical tube 10 and “rotation” of the medical tube 10 around an axis of the medical tube 10 can be prevented with certainty. As a result, it is possible to prevent the occurrence of a case that the medical tube securing jig 1 gives a patient a pain or a case where a defect occurs in connection between the medical tube 10 and equipment connected to the medical tube 10 (the blood pump 410 of the ventricular assist system, the controller 440 which controls the blood pump 410 or the like).

In the medical tube securing jig 1 according to the embodiment 1, because of the formation of the slit portion 212 in the sealing member 210, the sealing member 210 is exchangeable. Accordingly, when the sealing member 210 is deteriorated with time and an exchange of the sealing member 210 becomes necessary, the sealing member 210 can be exchanged.

In exchanging the sealing member 210, the waterproof cap 300 is pulled out from the sealing member pressing body 220, and is slid on the outer peripheral surface of the medical tube 10 to the position away from the sealing member pressing body 220. Then, the male threads 221 formed on the sealing member pressing body 220 are disengaged from the female thread 145 formed on the recessed portion 140, and the sealing member pressing body 220 is slid to a position away from the sealing member 210. The washer 230 is also moved away from the sealing member. In such a stat, the sealing member 210 is pulled out from the recessed portion 140, and the slit portion 212 of the sealing member 210 is expanded, and the sealing member 210 is removed from the outer peripheral surface of the medical tube 10. Then, a new sealing member is mounted on the medical tube 10.

In mounting the new sealing member 210, the slit portion 212 of the new sealing member 210 is expanded, the sealing member 210 is mounted on an outer peripheral surface side of the medical tube 10, and the sealing member 210 is fitted in the recessed portion 140 of the skin securing member 100. Then, the sealing member pressing body 220 is fastened to the recessed portion 140 by making the male threads 221 of the sealing member pressing body 220 threadedly engage with the female threads 145 of the recessed portion 140, thus applying a pressing force to the sealing member 210 by way of the washer 230 and, thereafter, the waterproof cap 300 is mounted on the sealing member pressing body 220. The new sealing member 210 can be mounted by performing such an operation.

As has been described heretofore, in the medical tube securing jig 1 according to the embodiment 1, as the mounting structure for mounting the sealing member pressing body 220 on the skin securing member 100, the structure is adopted where the sealing member pressing body 220 is mounted on the skin securing member 100 by making the male threads 221 formed on the sealing member pressing body 220 threadedly engage with the female threads 145 formed on the recessed portion 140 of the skin securing member 100 thus fastening the sealing member pressing body 220.

Accordingly, in the medical tube securing jig 1 according to the embodiment 1, a sufficient pressing force can be applied to the sealing member 210 and hence, the sealing member 210 can be elastically deformed in the recessed portion 140 of the skin securing member 100 without forming a gap. As a result, it is possible to bring the sealing member 210 close contact with the side wall surface 144 of the recessed portion 140, the bottom surface 142 of the recessed portion 140, and the outer peripheral surface of the medical tube 10 without forming a gap. At this stage of operation, the sealing member 210 is brought into close contact with the side wall surface 144 of the recessed portion 140, the bottom surface 142 of the recessed portion 140, and the outer peripheral surface of the medical tube 10 respectively in face contact.

In this manner, the sealing member 210 is brought into close contact with the side wall surface 144 of the recessed portion, the bottom surface 142 of the recessed portion 140, and the outer peripheral surface of the medical tube 10 respectively in face contact and hence, the medical tube securing jig 1 has high liquid sealing property at the outside of the body and the inside of the body. Accordingly, the medical tube securing jig 1 is brought into a sealed state having high liquid sealing property at the outside of the body and the inside of the body and hence, the invasion of the germs or the like into the body can be prevented with certainty whereby the occurrence of infection diseases can be prevented.

The sealing member 210 is brought into close contact with the side wall surface 144 of the recessed portion, the bottom surface 142 of the recessed portion 140, and the outer peripheral surface of the medical tube 10 respectively in face contact and hence, at the skin securing member 100, “displacement” of the medical tube 10 along an axial direction of the medical tube 10 and “rotation” of the medical tube 10 around an axis of the medical tube 10 can be prevented with certainty.

Further, the slit portion 212 is formed on the sealing member 210 and hence, the sealing member 210 is exchangeable. Accordingly, for example, even in the case where the sealing member 210 is deteriorated so that an exchange of the sealing member 210 becomes necessary, the sealing member 210 can be exchanged.

In the medical tube securing jig 1 according to the embodiment 1, among the constitutional parts (the sealing member 210, the washer 230, and the sealing member pressing body 220) of the medical tube holding mechanism part 200 of the medical tube securing jig 1, the sealing member 210 is disposed at a position closest to the inside of the body. Accordingly, the part which adheres to a skin which is a living tissue is only the sealing member 210 besides the skin securing member 100. Further, the sealing member 210 is elastically deformed by being pressed by the sealing member pressing body 220 so that the sealing member 210 is brought into a state where the sealing member 210 is embedded in the recessed portion 140. Accordingly, the sealing member 210 is brought into close contact with the side wall surface 144 of the recessed portion 140, the bottom surface 142 of the recessed portion 140, and the outer peripheral surface of the medical tube 10 in face contact and hence, the portion which is brought into contact with a living tissue is only a very small portion of the sealing member 210. Accordingly, an adhesion range of the sealing member 210 can be suppressed to a minimum range and hence, the sealing member 210 can be easily exchanged even when it is necessary to exchange the sealing member 210.

In the medical tube securing jig 1 of this embodiment 1, the sealing member 210 plays the role of holding the medical tube 10 such that the medical tube 10 does not move in the transdermal part, that is, the skin 500 besides the role of providing sealing between the outside of the body and the inside of the body with high liquid sealing property. Accordingly, it is sufficient to provide only the sealing member 210, the sealing member pressing body 220 which presses the sealing member 210 and the washer 230 as the constitutional parts which form the medical tube holding mechanism part 200 of the medical tube securing jig 1 whereby the structure of the medical tube securing jig 1 can be simplified, and the number of parts can be reduced.

### Embodiment 2

FIG. 7 is a view for describing constitutional parts of a medical tube securing jig 1A according to an embodiment 2 as individual parts. FIG. 7 is a view corresponding to FIG. 3 used in the description of the medical tube securing jig 1 according to the embodiment 1. In FIG. 7, a portion expressed by (a) indicates a skin securing member (referred to as “skin securing member 100A” in the medical tube securing jig 1A of the embodiment 2), and a portion expressed by (b) indicates constitutional parts (a sealing member 210, a washer 230 and a sealing member pressing body 220) of a medical tube holding mechanism part 200, and a portion expressed by (c) indicates a waterproof cap 300.

In the medical tube securing jig 1A according to the embodiment 2, the configuration of parts of the skin securing member 100A expressed by (a) in FIG. 7 differs from the skin securing member 100 of the medical tube securing jig 1 according to the embodiment 1 (the portion expressed by (a) in FIG. 3). However, the configuration of the medical tube holding mechanism part 200 which is the portion expressed by (b) in FIG. 7 and the configuration of the waterproof cap 300 which is the portion expressed by (c) in FIG. 7 are substantially equal to those of the medical tube holding mechanism part 200 of the medical tube securing jig 1 according to the embodiment 1 (the portion expressed by (b) in FIG. 3) and the configuration of the waterproof cap 300 (see the portion expressed by (c) in FIG. 3). Accordingly, with respect to the respective constitutional elements of the medical tube securing jig 1A according to the embodiment 2, the constitutional elements identical with the constitutional elements of the medical tube securing jig 1 according to the embodiment 1 are given with the same symbols, and the repeated explanation of these constitutional elements is omitted as much as possible.

Hereinafter, the medical tube securing jig 1A according to the embodiment 2 is described. In this embodiment 2, the skin securing member 100A is mainly described.

In the same manner as the skin securing member 100 of the medical tube securing jig 1 according to the embodiment 1, the skin securing member 100A has, as the constitutional elements thereof, a flange portion (referred to as “flange portion 110A” in the medical tube securing jig 1A according to the embodiment 2), a protruding body 120 (including a body inside protruding body 120a), and a brim portion 130. The flange portion 110A which the skin securing member 100A has is made of porous metal having biocompatibility. For example, titanium in a sponge form can be used as the porous metal. However, the porous metal is not limited to such titanium.

In the medical tube securing jig 1A according to the embodiment 2, the flange portion 110A made of porous metal (hereinafter also simply abbreviated as “flange portion 110A”) and the protruding body 120 are formed as separate parts. Also in the medical tube securing jig 1A according to the embodiment 2, the protruding body 120 is formed so as to protrude not only toward the outside of the body but also toward the inside of the body, and a portion of the protruding body 120 protruding toward the inside of the body is referred to as “body inside protruding body 120a”. Accordingly, the flange portion 110A is joined to the body inside protruding body 120a.

That is, a space portion 111 which allows the body inside protruding body 120a to pass therethrough is formed at a center portion of the flange portion 110A, and the flange portion 110A and the body inside protruding body 120a are joined to each other in a state where the body inside protruding body 120a is made to pass through the space portion 111 formed in the flange portion 110A. In the medical tube securing jig 1A according to the embodiment 2, the flange portion 110A and the body inside protruding body 120a are joined to each other by an adhesive agent, welding or sinter bonding. The skin securing member 100A is formed by joining the flange portion 110A and the body inside protruding body 120a to each other.

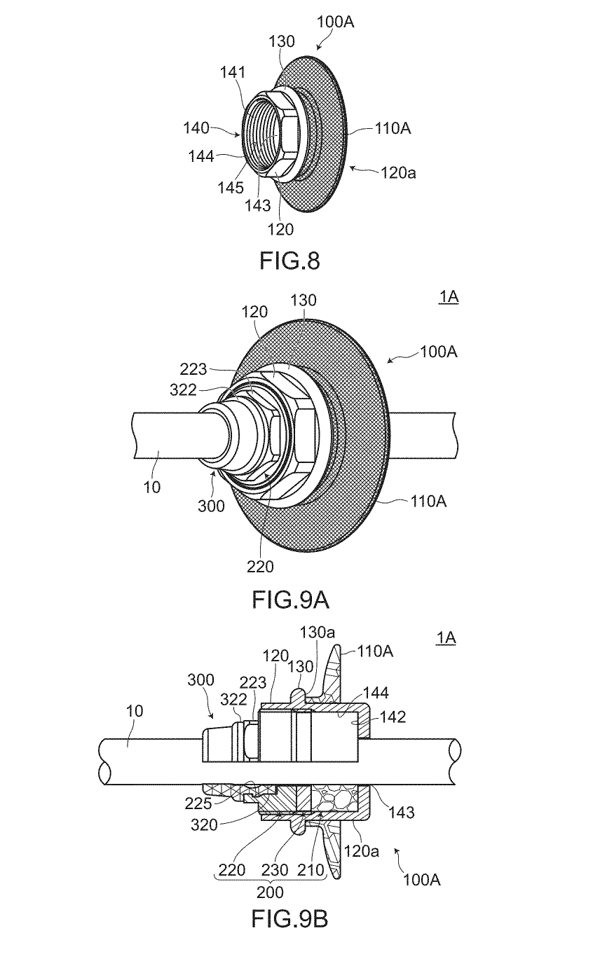

FIG. 8 is a view showing the skin securing member 100A in a state where the flange portion 110A and the body inside protruding body 120a are joined to each other. In an assembling step of the medical tube securing jig 1A according to the embodiment 2 using the skin securing member 100A shown in FIG. 8, the medical tube securing jig 1A can be assembled in accordance with steps (first step to fifth step) substantially equal to the flowchart shown in FIG. 4 described with respect to the medical tube securing jig 1 according to the embodiment 1. Accordingly, the description is omitted with respect to the assembling steps of the medical tube securing jig 1A according to the embodiment 2 which are performed after the skin securing member 100A shown in FIG. 8 is formed by joining the flange portion 110A and the body inside protruding body 120a to each other.

FIG. 9A and FIG. 9B are views showing the medical tube securing jig 1A according to the embodiment 2 in a state where the medical tube securing jig 1A is assembled in accordance with the respective steps shown in FIG. 4. FIG. 9A is a perspective view and FIG. 9B is a plan view showing a main part in cross section. FIG. 9A and FIG. 9B are views corresponding to FIG. 5A and FIG. 5B showing the medical tube securing jig 1 according to the embodiment 1. In an external appearance, the medical tube securing jig 1A according to the embodiment 2 differs from the medical tube securing jig 1 according to the embodiment 1 (see FIG. 5A and FIG. 5B) with respect to a point that a flange portion 110A made of a porous material is formed in place of the flange portion 110 shown in FIG. 5A and FIG. 5B, and is substantially equal to the medical tube securing jig 1 according to the embodiment 1 with respect to other constitutional elements.

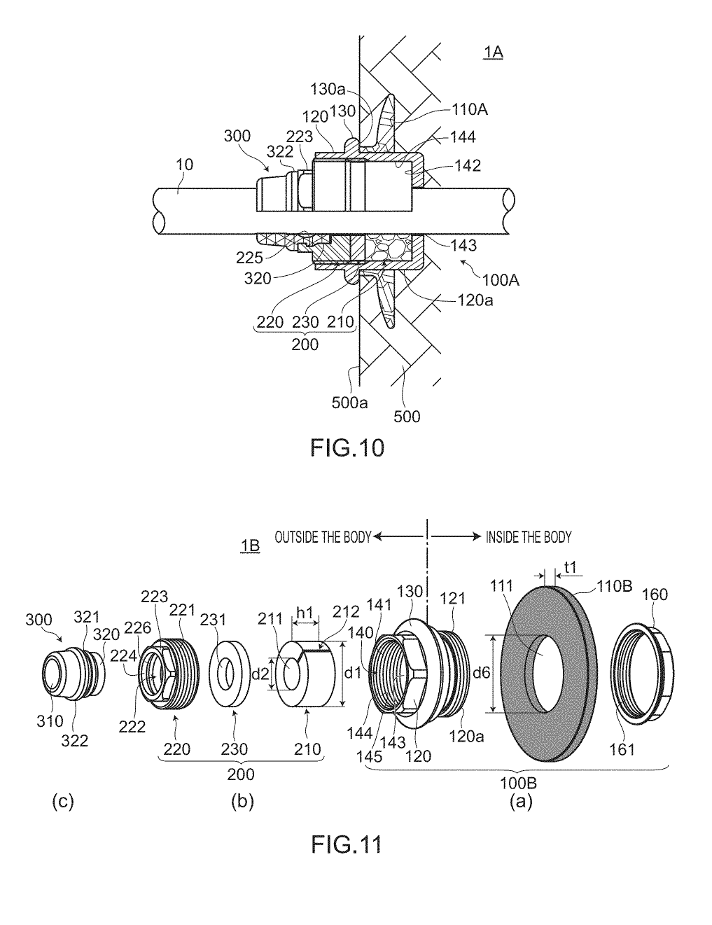

FIG. 10 is a cross-sectional view of a main part showing a state where the medical tube securing jig 1A according to the embodiment 2 is fixed to a transdermal part, that is, a skin 500. The state where the medical tube securing jig 1A is fixed to the skin 500 means a state where the skin securing member 100A is fixed to the skin 500 due to adhesion between the skin securing member 100A and a part of the skin 500 which is a living tissue.

As shown in FIG. 10, the skin securing member 100A of the medical tube securing jig 1A according to the embodiment 2 is fixed to the skin 500 so that a state is brought about where the medical tube 10 is fixed to the skin 500. That is, the medical tube 10 is held on the skin securing member 100 in a state where the movement of the medical tube 10 is prevented by constitutional parts (the sealing member 210, the washer 230, and the sealing member pressing body 220) of the medical tube holding mechanism part 200. Accordingly, a state is brought about where the medical tube 10 is fixed to the skin 500.

In the medical tube securing jig 1A according to the embodiment 2, the flange portion of the skin securing member 100A is formed of the flange portion 110A made of a porous metal. Accordingly, in addition to an advantageous effects acquired by the medical tube securing jig 1 according to the embodiment 1, the medical tube securing jig 1A according to the embodiment 2 acquires an advantageous effect brought about by the use of the flange portion 110A made of porous metal. That is, with the use of the flange portion 110A made of porous metal, a tissue of a skin infiltrates into a large number of pores formed in the flange portion 110A and hence, the tissue of the skin and the flange portion 110A easily adhere to each other. Accordingly, the invasion of germs or the like into a living body can be prevented whereby it is possible to increase an effect of suppressing the occurrence of infectious diseases.

The flange portion 110A may be formed such that a large number of fine pores are formed two-dimensionally (in a planar layered structure). However, the flange portion 110A may be formed such that a porous layer is formed where fine pores communicate with each other three-dimensionally (stereoscopically). In this manner, the porous layer where the fine pores communicate with each other three-dimensionally (stereoscopically) is formed in the flange portion 110A and hence, a tissue of a skin and the flange portion 110A adhere with each other with more certainty thus further enhancing an effect of suppressing the occurrence of infectious diseases.

### Embodiment 3

FIG. 11 is a view for describing constitutional parts of a medical tube securing jig 1B according to an embodiment 3 as individual parts. FIG. 11 is a view corresponding to FIG. 3 used in the description of the medical tube securing jig 1 according to the embodiment 1. A portion expressed by (a) indicates a skin securing member (referred to as “skin securing member 100B” in the medical tube securing jig 1B of the embodiment 3), a portion expressed by (b) indicates constitutional parts (a sealing member 210, a washer 230 and a sealing member pressing body 220) of a medical tube holding mechanism part 200, and a portion expressed by (c) indicates a waterproof cap 300.

In the medical tube securing jig 1B according to the embodiment 3, the configuration of parts of the skin securing member 100B expressed by (a) in FIG. 11 differ from the corresponding parts of the skin securing member 100 of the medical tube securing jig 1 according to the embodiment 1 (the portion expressed by (a) in FIG. 3). However, the configuration of the medical tube holding mechanism part 200 which is the portion expressed by (b) in FIG. 11 and the configuration of the waterproof cap 300 which is the portion expressed by (c) in FIG. 11 are substantially equal to the parts expressed by (b) and (c) in FIG. 3 in the medical tube securing jig 1 according to the embodiment 1. Accordingly, with respect to the respective constitutional elements of the medical tube securing jig 1B according to the embodiment 3, the constitutional elements identical with the constitutional elements of the medical tube securing jig 1 according to the embodiment 1 are given with the same symbols, and the repeated explanation of these constitutional elements is omitted as much as possible.

Hereinafter, the medical tube securing jig 1B according to the embodiment 3 is described. In this embodiment 3, the skin securing member 100B is mainly described.

In the same manner as the skin securing member 100 of the medical tube securing jig 1 according to the embodiment 1, the skin securing member 100B has, as the constitutional elements thereof, a flange portion (referred to as “flange portion 110B” in the medical tube securing jig 1B according to the embodiment 3), a protruding body 120 (including a body inside protruding body 120a), and a brim portion 130.

The flange portion 110B which the skin securing member 100B has is made of a non-woven fabric having biocompatibility. For example, as such a non-woven fabric having biocompatibility, polytetrafluoroethylene (PTFE) felt known in a medical field can be exemplified. Such PTFE felt has the structure where fibers which form the PTFE are entangled with each other, and a large number of fine gaps are formed between the entangled fibers.

Also in the medical tube securing jig 1B according to the embodiment 3, the flange portion 110B and the protruding body 120 are formed as separate parts. Also in the medical tube securing jig 1B according to the embodiment 3, a portion of the protruding body 120 which protrudes toward the inside of the body is referred to as “body inside protruding body 120a”. Accordingly, the flange portion 110B is joined to the body inside protruding body 120a.

That is, a space portion 111 which allows the body inside protruding body 120a to pass therethrough is formed at a center portion of the flange portion 110B, and the flange portion 110B and the body inside protruding body 120a are joined to each other in a state where the body inside protruding body 120a is made to pass through the space portion 111 formed in the flange portion 110B. The medical tube securing jig 1B according to the embodiment 3 has a fixing nut 160 for fixing the flange portion 110B to the body inside protruding body 120a. On the other hand, male threads 121 which are threadedly engageable with female threads 161 formed on the fixing nut 160 are formed on an outer peripheral surface of the body inside protruding body 120a.

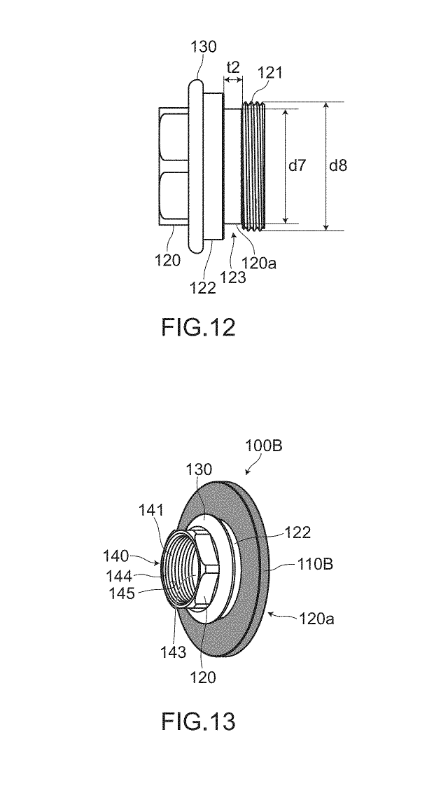

FIG. 12 is a plan view of the protruding body 120 of the medical tube securing jig 1B according to the embodiment 3 in the form that the protruding body 120 is taken out from the medical tube securing jig 1B. As shown in FIG. 12, with respect to the protruding body 120 in the medical securing jig 1B according to the embodiment 3, the male threads 121 are formed on a distal end portion (the distal end portion directed toward the inside of the body) of the body inside protruding body 120a of the protruding body 120. On the other hand, a flange pressing portion 122 is provided for sandwiching the flange portion 110B between the flange pressing portion 122 and the fixing nut 160 is formed on the body inside protruding body 120a on a brim portion 130 side. A diameter d6 of the space portion 111 formed in the flange portion 110B (see FIG. 11) is set substantially equal to an outer diameter d7 of the body inside protruding body 120a. Further, an outer diameter d8 of the male threads 121 is set slightly larger than the diameter d6 of the space portion 111 formed in the flange portion 110.

Between the flange pressing portion 122 and male threads 121 of the body inside protruding body 120a, a zone where the male threads 121 are not formed (referred to as male threads non-forming zone t2) exists in a zone corresponding to a thickness t1 of the flange portion 110B. An outer diameter of the male threads non-forming zone t2 of the body inside protruding body 120a (the outer diameter d7 of the body inside protruding body 120a) is small compared to the outer diameter d8 of the male threads 121 and an outer diameter of the flange pressing portion 122 (not shown in the drawing) thus forming a recessed groove 123 along a circumferential direction.

The flange portion 110B and the body inside protruding body 120a respectively adopt the above-mentioned configurations and hence, the body inside protruding body 120a is made to pass through the space portion 111 formed in the flange portion 110B. That is, the flange portion 110B is made of a non-woven fabric (in this case, a PTFE felt) which is a soft material. Accordingly, the male threads 121 formed on the body inside protruding body 120a can pass thorough the space portion 111 in such a manner that the male threads 121 expand by pressing the circular space portion 111 formed on the flange portion 110B.

By making the male threads 121 formed on the body inside protruding body 120a pass through the circular space portion 111 formed in the flange portion 110B in this manner, a state is brought about a peripheral surface of the flange portion 110B on a space portion 111 side enters the recessed groove 123 formed on the body inside protruding body 120a. By making the female threaded portion 161 of the fixing nut 160 threadedly engage with the male threaded portion 121 of the body inside protruding body 120a thus fastening the fixing nut 160 and hence, the flange portion 110B and the body inside protruding body 120a can be joined to each other.

In joining the flange portion 110B and the body inside protruding boy 120a to each other, it is preferable to fasten a peripheral surface of the space portion 111 of the flange portion 110B, the recessed groove 123 of the body inside protruding body 120a, and the fixing nut 161 together by making the female threaded portion 161 of the fixing nut 160 threadedly engage with the male threaded portion 121 of the body inside protruding body 120a in a state where an adhesive agent is applied by coating to the peripheral surface of the space portion 111 of the flange portion 110B, the recessed groove 123 of the body inside protruding body 120a, and the fixing nut 161. Accordingly, loosening of the fixing nut 160 can be prevented and hence, the flange portion 110B and the body inside protruding body 120a can be joined to each other with more certainty. The flange portion 110B and the body inside protruding body 120a can be joined to each other in this manner. The structure formed by joining the flange portion 11B and the body inside protruding body 120a to each other forms the skin securing member 100B.

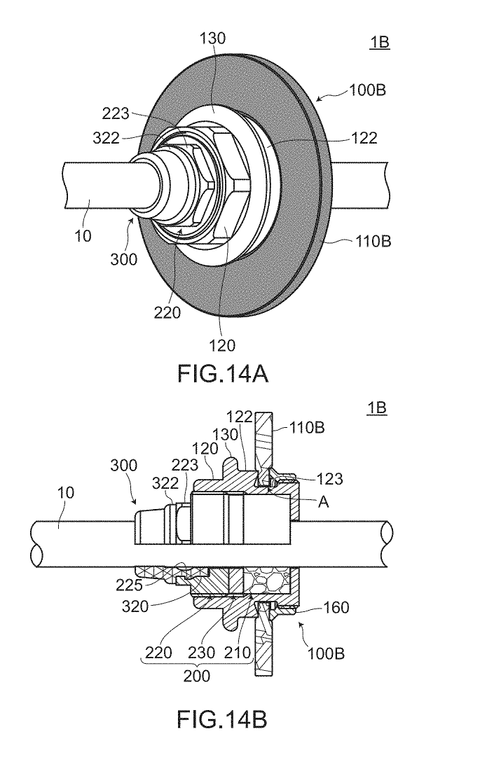

FIG. 13 is a view showing the skin securing member 100B in a state where the flange portion 110B and the body inside protruding body 120a are joined to each other. In a state where the flange portion 110B and the body inside protruding body 120a are joined to each other, a state is brought about where the flange portion 110B is pressed and compressed by fastening of the fixing nut 160. Accordingly, a thickness of a portion of the flange portion 110B sandwiched between the fixing nut 160 and the flange pressing portion 122 (see an arrow A shown in FIG. 14B) becomes smaller than a thickness of a remaining portion of the flange portion 110B.

In an assembling step of the medical tube securing jig 1B according to the embodiment 3 using the skin securing member 100B shown in FIG. 13, the medical tube securing jig 1B can be assembled in accordance with steps (first step to fifth step) substantially equal to the flowchart shown in FIG. 4 described with respect to the medical tube securing jig 1 according to the embodiment 1. Accordingly, the description is omitted with respect to the assembling steps of the medical tube securing jig 1B according to the embodiment 3 which are performed after the skin securing member 100B shown in FIG. 13 is formed by joining the flange portion 110B and the body inside protruding body 120a to each other.

FIG. 14A and FIG. 14B are views showing the medical tube securing jig 1B according to the embodiment 3 in a state where the medical tube securing jig 1B is assembled in accordance with the respective steps shown in FIG. 4. FIG. 14A is a perspective view, and FIG. 14B is a plan view showing a main part in cross section. FIG. 14A and FIG. 14B are views corresponding to FIG. 5A and FIG. 5B showing the medical tube securing jig 1 according to the embodiment 1. In an external appearance, the medical tube securing jig 1B according to the embodiment 3 differs from the medical tube securing jig 1 according to the embodiment 1 (see FIG. 5A and FIG. 5B) with respect to a point that a flange portion 110B made of a non-woven fabric (in this case, a PTFE felt) is formed in place of the flange portion 110 shown in FIG. 5A and FIG. 5B, and is substantially equal to the medical tube securing jig 1 according to the embodiment 1 (see FIG. 5A and FIG. 5B) with respect to other constitutional elements.

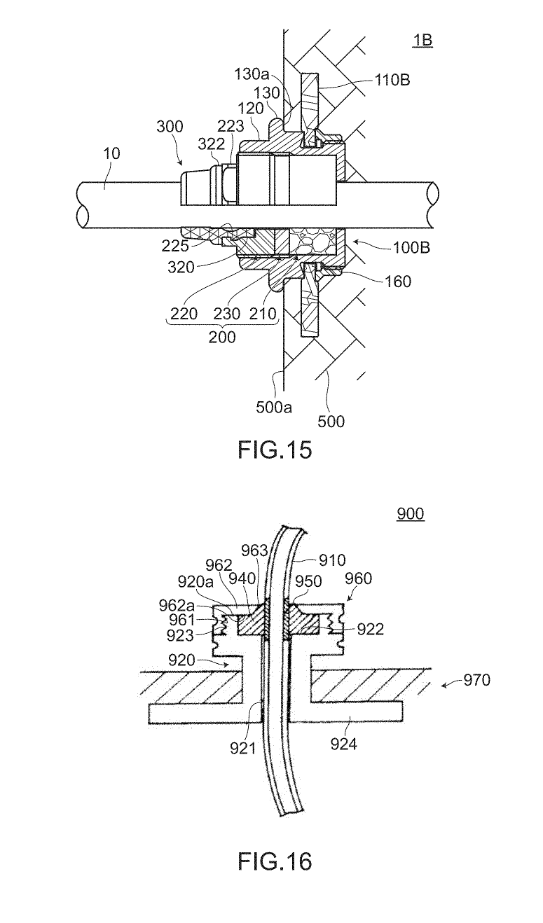

FIG. 15 is a cross-sectional view of a main part showing a state where the medical tube securing jig 1B according to the embodiment 3 is fixed to a transdermal part, that is, a skin 500. The state where the medical tube securing jig 1B is fixed to the skin 500 means a state where the skin securing member 100B is fixed to the skin 500 due to adhesion between the skin securing member 100 and a part of the skin 500 which is a living tissue.

As shown in FIG. 15, the skin securing member 100B of the medical tube securing jig 1B according to the embodiment 3 is fixed to the skin 500 so that a state is brought about where the medical tube 10 is fixed to the skin 500. That is, the medical tube 10 is held on the skin securing member 100 in a state where the movement of the medical tube 10 is prevented by constitutional parts (the sealing member 210, the washer 230, and the sealing member pressing body 220) of the medical tube holding mechanism part 200. Accordingly, a state is brought about where the medical tube 10 is fixed to the skin 500.

In the medical tube securing jig 1B according to the embodiment 3, the flange portion of the skin securing member 100B is formed of the flange portion 110B made of a non-woven fabric(in this case, a PTFE felt) having biocompatibility. Accordingly, in addition to an advantageous effects acquired by the medical tube securing jig 1 according to the embodiment 1, the medical tube securing jig 1B according to the embodiment 3 acquires an advantageous effect brought about by the use of the flange portion 110B made of a non-woven fabric (in this case, a PTFE felt) having biocompatibility.

That is, with the use of the flange portion 110B made of a PTFE felt, a tissue of a skin infiltrates into gaps in fibers which form the PTFE felt and hence, the tissue of the skin and the flange portion 110B easily adhere to each other. Accordingly, the invasion of germs or the like into a living body can be prevented whereby it is possible to increase an effect of suppressing the occurrence of infectious diseases. Further, the flange portion 110B is made of a soft material such as a non-woven fabric (in this case, a PTFE felt) having biocompatibility and hence, it is also possible to acquire an advantageous effect that a burden imposed on a body can be suppressed when the flange portion is embedded in the skin 500.

The present invention is not limited to the above-mentioned respective embodiments, and various modifications are conceivable without departing the gist of the present invention. For example, the following modifications are also conceivable.
(1) The case is exemplified where the medical tube securing jigs 1, 1A, 1B according to the above-mentioned respective embodiments are used in a ventricular assist system. However, the medical tube securing jigs 1, 1A, 1B according to the respective embodiments can be used in applications other than a ventricular assist system. Accordingly, the medical tube 10 is not limited to a drive line (a cable connecting a blood pump and a controller to each other) used in a ventricular assist system, and includes medical tubes in general which are necessary to be fixed to a skin part when the medical tube is made to pass through from the outside of the body to the inside of the body.
(2) The case is exemplified where, in the skin securing members 100, 100A, 100B of the medical tube securing jigs 1, 1A, 1B according to the above-mentioned respective embodiments, an external appearance of the flange portion 110, 110A, 110B has a disc-like shape. However, the external appearance of each flange portion may have an elongated circular shape. Further, the case is exemplified where, in the medical tube securing jigs 1, 1A, 1B according to the respective embodiments, the skin securing member 100, 100A, 100B adopts the configuration where the protruding body 120 protrudes in a direction orthogonal to each flange portion 110, 110A, 110B. Accordingly, the medical tube 10 is mounted in a direction orthogonal to the flange portion 110, 110A, 110B. However, the medical tube 10 may be mounted in an oblique direction with respect to each flange portion 110, 110A, 110B so as to make the protruding body 120 protrude in an oblique direction with respect to each flange portion 110, 110A, 110B.
(3) The case is exemplified where, in the medical tube securing jig 1A according to the above-mentioned embodiment 2, the flange portion 110A made of porous metal is joined to the body inside protruding body 120a by an adhesive agent, welding, or sinter bonding. However, the present invention is not limited to such a case. For example, in the same manner as the case of the medical tube securing jig 1B according to the embodiment 3, the flange portion 110A may be joined to the body inside protruding body 120a by forming the male threads 121 on the outer peripheral surface of the body inside protruding body 120a and by fastening the fixing nut 160 by making the female threads 161 formed on the fixing nut 160 threadedly engage with the male threads 121 formed on the outer peripheral surface of the body inside protruding body 120a. In this case, it is preferable to use an adhesive agent together with such fastening configuration.
(4) In the above-mentioned respective embodiments, a pipe formed of a hard member made of a hard resin or the like may be mounted on the outer peripheral surface of the medical tube 10 in a close contact state between the medical tube 10 and the sealing member 210. With such a configuration, in the case where the medical tube 10 is formed of a member which is easily deformed by pressing, it is possible to prevent the deformation of the medical tube when the sealing member 210 is pressed and deformed.

### REFERENCE SIGNS LIST

1, 1A, 1B: medical tube securing jig

10: medical tube

100, 100A, 100B: skin securing member

110, 110A, 100B: flange portion

111: space portion

120: protruding body

120a: body inside protruding body

121: male thread

122: flange pressing portion

123: recessed groove

130: brim portion

140: recessed portion (recessed portion of skin securing member 100)

141: opening

142: bottom surface

143: through hole (first through hole)

144: side wall surface

145: female thread

160: fixing nut

161: female thread

200: medical tube holding mechanism part

210: sealing member

211: through hole (second through hole)

212: slit portion

220: sealing member pressing body

221: male thread

222: through hole (third through hole)

223: screw head

224: recessed portion (recessed portion of sealing member pressing body 220)

225: waterproof cap inserting gap

226: recessed groove

230: washer

231: through hole (fourth through hole)

300: waterproof cap

310: through hole (fifth through hole)

320: gap insertion portion

321: projecting portion

322: brim portion

400: ventricular assist system

410: blood pump

440: controller

500: skin (transdermal part)

1-10. (canceled)

11. A medical tube securing jig for securing a medical tube to a transdermal part in inserting the medical tube from an outside of a body to an inside of the body, the medical tube securing jig comprising:
a skin securing member having a flange portion which is embedded in the transdermal part and a cylindrical protruding body which protrudes from the flange portion at least toward the outside of the body; and
a medical tube holding mechanism part which is mounted in a state where the medical tube holding mechanism part is inserted into the protruding body of the skin securing member so as to hold the medical tube, wherein
a recessed portion whose opening is directed toward the outside of the body is formed on the protruding body of the skin securing member, a first through hole which allows the medical tube to pass therethrough is formed in a bottom surface of the recessed portion in a state where the first through hole penetrates the bottom surface toward the inside of the body and female threads are formed on a side wall surface of the recessed portion between the opening and the bottom surface,
the medical tube holding mechanism part includes:
a sealing member made of a material which has elasticity and liquid sealing property, the sealing member having a second through hole which allows the medical tube to pass therethrough, the sealing member being engageable with the recessed portion by fitting engagement in a state where the medical tube is made to pass through the second through hole, the sealing member being elastically deformed when a pressing force in a direction from the outside of the body to the inside of the body is applied to the sealing member in a state where the sealing member engages with the recessed portion by fitting engagement thus being brought into close contact with a side wall surface of the recessed portion, the bottom surface of the recessed portion, and the medical tube; and
a sealing member pressing body disposed at a position more outside of the body than the sealing member, the sealing member pressing body having male threads being threadedly engageable with the female threads formed on the recessed portion, the sealing member pressing body having a third through hole which allows the medical tube to pass therethrough along a center axis of the male threads, the sealing member pressing body being configured to apply the pressing force to the sealing member in fastening the sealing member pressing body by making the male threads engage with the female threads in a state where the medical tube is made to pass through the third through hole, wherein
a slit portion is formed in the sealing member, the slit portion reaching the other end side from one end side of the sealing member along a longitudinal direction of the medical tube, the slit portion reaching the second through hole in a radial direction.

12. The medical tube securing jig according to claim 11, wherein
the medical tube holding mechanism part has a washer interposed between the sealing member and the sealing member pressing body, and the washer is made of a material where a frictional force between the washer and the sealing member pressing body is smaller than a frictional force between the washer and the sealing member.

13. The medical tube securing jig according to claim 11, wherein
the skin securing member further has a brim portion which is disposed at a position on a more outside of the body than the flange portion and spaced apart from the flange portion, and
the brim portion is disposed such that a surface of the brim portion directed toward the inside of the body is in contact with a surface of the transdermal part on the outside of the body in a state where the skin securing member is fixed to the transdermal part.

14. The medical tube securing jig according to claim 11, wherein
the flange portion is made of porous metal having biocompatibility.

15. The medical tube securing jig according to claim 14, wherein
the flange portion made of porous metal and the protruding body are formed as separate parts from each other, and
the protruding body is disposed so as to protrude not only toward the outside of the body but also toward the inside of the body,
assuming a portion of the protruding body protruding toward the inside of the body is a body inside protruding body, a space portion which allows the body inside protruding body to pass therethrough is formed on a center portion of the flange portion made of the porous metal, and the flange portion made of the porous metal and the body inside protruding body are joined to each other in a state where the body inside protruding body is made to pass through the space portion.

16. The medical tube securing jig according to claim 11, wherein
the flange portion is made of a non-woven fabric having biocompatibility.

17. The medical tube securing jig according to claim 16, wherein the flange portion which is made of the non-woven fabric and the protruding body are formed as separate parts from each other, and
the protruding body is disposed so as to protrude not only toward the outside of the body but also toward the inside of the body, and
assuming a portion of the protruding body protruding toward the inside of the body is a body inside protruding body, a space portion which allows the body inside protruding body to pass therethrough is formed on a center portion of the flange portion made of the non-woven fabric, and the flange portion made of the non-woven fabric and the body inside protruding body are joined to each other in a state where the body inside protruding body is made to pass through the space portion.

18. The medical tube securing jig according to claim 17, wherein
polytetrafluoroethylene (PTFE) felt is used as the non-woven fabric.

19. The medical tube securing jig according to claim 11, further comprising a waterproof cap which is mounted on an end portion of the sealing member pressing body on the outside of the body in addition to the skin securing member and the medical tube holding mechanism part, wherein
the waterproof cap is made of a material having elasticity and liquid sealing property, the waterproof cap having a gap insertion portion which is insertable into a gap formed between the sealing member pressing body and the medical tube so as to fill the gap, and the waterproof cap being annularly mounted on the medical tube such that the waterproof cap is slidable along a longitudinal direction of the medical tube in a state where the waterproof cap is brought into close contact with an outer peripheral surface of the medical tube.

20. The medical tube securing jig according to claim 12, wherein
the flange portion is made of porous metal having biocompatibility.

21. The medical tube securing jig according to claim 20, wherein
the flange portion made of porous metal and the protruding body are formed as separate parts from each other, and
the protruding body is disposed so as to protrude not only toward the outside of the body but also toward the inside of the body,
assuming a portion of the protruding body protruding toward the inside of the body is a body inside protruding body, a space portion which allows the body inside protruding body to pass therethrough is formed on a center portion of the flange portion made of the porous metal, and the flange portion made of the porous metal and the body inside protruding body are joined to each other in a state where the body inside protruding body is made to pass through the space portion.

22. The medical tube securing jig according to claim 12, wherein
the flange portion is made of a non-woven fabric having biocompatibility.

23. The medical tube securing jig according to claim 22, wherein
the flange portion which is made of the non-woven fabric and the protruding body are formed as separate parts from each other, and
the protruding body is disposed so as to protrude not only toward the outside of the body but also toward the inside of the body, and
assuming a portion of the protruding body protruding toward the inside of the body is a body inside protruding body, a space portion which allows the body inside protruding body to pass therethrough is formed on a center portion of the flange portion made of the non-woven fabric, and the flange portion made of the non-woven fabric and the body inside protruding body are joined to each other in a state where the body inside protruding body is made to pass through the space portion.

24. The medical tube securing jig according to claim 23, wherein
polytetrafluoroethylene (PTFE) felt is used as the non-woven fabric.

25. The medical tube securing jig according to claim 11, wherein
the medical tube is a cable which connects a blood pump disposed in the inside of the body and a controller disposed on the outside of the body to each other in a ventricular assist system.

26. The medical tube securing jig according to claim 11, wherein
the flange portion is made of porous metal having biocompatibility, and
the medical tube is a cable which connects a blood pump disposed in the inside of the body and a controller disposed on the outside of the body to each other in a ventricular assist system.

27. The medical tube securing jig according to claim 12, wherein
the medical tube is a cable which connects a blood pump disposed in the inside of the body and a controller disposed on the outside of the body to each other in a ventricular assist system.
